(19) United States
(12) Patent Application Publication
(10) Pub. No.: US 2021/0177953 A1
(43) Pub. Date: Jun. 17, 2021

(54) Engineered Non-Human Derived Immune Cells for Universal Adoptive Antigen Cellular Immunotherapy

(71) Applicant: Asclepius Therapy LLC, Pomona, NY (US)

(72) Inventor: Xiaohua Wang, Pomona, NY (US)

(21) Appl. No.: 16/759,888

(22) PCT Filed: Jul. 20, 2018
(86) PCT No.: PCT/US2018/043040

§ 371 Date: Apr. 28, 2020

Related U.S. Application Data
(60) Provisional application No. 62/585,593, filed on Nov. 14, 2017.

(51) Int. Cl. A61K 39/00; C07K 14/725; C07K 16/28

(52) U.S. Cl. CPC A61K 39/001112; C07K 14/7051; C07K 16/2803; C07K 2319/33; C07K 2317/622; C07K 2319/03; A61K 2039/5156

(57) ABSTRACT

This invention relates to non-human immune cells for universal chimeric antigen receptor and related immune cell therapies.

### CROSS REFERENCE TO RELATED APPLICATION

This application claims priority to U.S. Provisional Application No. 62/585,593 filed on Nov. 14, 2017. The content of the application is incorporated herein by reference in its entirety.

### FIELD OF THE INVENTION

This invention relates to non-human immune cells for universal chimeric antigen receptor and related immune cell therapies.

### BACKGROUND OF THE INVENTION

Cell-based immunotherapy has emerged as a promising therapy with curative potential for the treatment of cancer and other major human diseases. T cells and other immune cells may be modified to target tumor antigens through the introduction of genetic material coding for artificial or synthetic receptors for antigen, termed Chimeric Antigen Receptors (CARs), specific to selected antigens. Targeted T cell therapy using CARs (CAR T) has shown recent clinical success in treating hematologic malignancies. So far all immune cell therapies against cancer, infectious diseases, or autoimmune diseases use immune cells derived from patients or healthy human donors as starting manufacture materials. Manufacture process including cell expansion and cell engineering ex vivo.

Despite the promises, various logistical obstacles limit widespread application of CAR-T immunotherapy. For instances, it takes weeks to prepare CAR. T cells; the process requires apheresis of autologous T cells, genetic modification and expansion of these cells, and several other steps before they can finally be reintroduced into a patient. This lengthy manufacturing process can be problematic because many patients may suffer from fulminant relapses that do not allow safe delays for cell manipulation and expansion of CAR T cells. Thus, there is an unmet need for universal CAR based immune cell therapy.

### SUMMARY OF INVENTION

This invention addresses the above mentioned unmet need by providing non-human derived immune cells for universal chimeric antigen receptor and related immune cell therapies.

In one aspect, the invention provides an engineered, non-human (animal derived) immune cell to deliver immunotherapy in the format of adoptive cellular therapy. The cell can comprise an antigen-binding receptor, e.g., a T cell antigen receptor (TCR) or chimeric antigen receptor (CAR) or a nucleic acid encoding the antigen-binding receptor. The TCR or CAR comprises an antigen binding domain, a transmembrane domain, and an activation domain. The engineered, non-human immune cell has one or more of the following features: (a) expressing a pro-function factor or comprising a nucleic acid encoding the pro-function factor; (b) lacking expression of a rejection factor; (c) expressing a graft sustaining factor or comprising a nucleic acid encoding the graft sustaining factor; (d) eliminating or modifying the expression of a host targeting factor, and (e) lacking the activity of an infection-risk factor.

Examples of the pro-function factor include human or humanized perforin, granzyme B, and pro-inflammatory cytokines. Examples of the rejection factor include galactose-α1,3-galactose or N-glycolylneuraminic acid (NeuGc). Examples of the graft sustaining factor include human complement-regulatory protein, CTLA4, CTLA4-Ig, PD-L1, a mutant form of human MHC class II, a wild type or mutant form of human MHC class I (e.g., HLA-E, HLA-G, HLA Cw3), CD47, HO-1, ATPase and thrombomdulin. Examples of the human complement-regulatory protein include CD55, CD46, and CD59. In some embodiments, an endogenous antigen receptor or activating receptor that recognizes a health human tissue is eliminated, modified or replaced in the cell. In some embodiments, one or more key molecules controlling the activity of human sensitive infectious reagents are eliminated in the cell for safety as well.

The engineered, non-human immune cell can be derived from a non-human animal or an in vitro cell line. The immune cell can be a CD4 or CD8 T cell, a Gamma-delta T cell, a NKT cell, a B cell, a NK cell, or a macrophage. The engineered, non-human immune cell can be engineered through germline modification, somatic modification, or ex vivo.

In the engineered, non-human immune cell, the T cell antigen receptor can be a naturally existing T cell receptor (TCR) specific for targeted antigen or its derivative(s). In some embodiments, the TCR binds to an antigen epitope on a tumor, such as an antigen associated with a hematologic malignancy or with a solid tumor. In some other embodiments, the TCR binds to an antigen epitope on or associated with an infectious pathogen, such as pathogenic parasite, bacterium or virus. In some other embodiments, the TCR binds to a self-antigen epitope on or associated with an autoimmune disease. In some other embodiments, the antigen binding domain binds to an antigen epitope that is labeled onto target cell pharmatheutically.

In the engineered, non-human immune cell, the antigen binding domain can be an antibody, an antigen-binding fragment thereof (such as a Fab or a scFv), or a ligand of the antigen.

In the engineered, non-human immune cell, the antigen binding domain can be an extracellular domain of a natural receptor or its derivatives, such as CD16, killer cell Ig-like receptors.

In some embodiments, the antigen binding domain binds to an antigen on a tumor, such as an antigen associated with a hematologic malignancy or with a solid tumor, or an antigen labeled onto a tumor. In some other embodiments, the antigen binding domain binds to an antigen on or associated with an infectious pathogen, such as pathogenic parasite, bacterium or virus. In some other embodiments, the antigen-binding domain binds to a self-antigen on or associated with an autoimmune disease. In some other embodiments, the antigen-binding domain binds to an antigen that is labeled onto target cell pharmatheutically.

In the engineered, non-human immune cell, the transmembrane domain can be a transmembrane portion of a transmembrane protein proven suitable in the CAR by state-of-art at the time.

In the engineered, non-human immune cell, the activation domain can be an activating motif or a combination of multiple activating motif, such as CD28 intracellular domain, 4-1BB intracellular domain, TCRζ. In some other embodiments, the activation domain activates T cell. In some other embodiments, the activation domain activates B cell. In some other embodiments, the activation domain activates NK cell. In some other embodiments, the activation domain activates macro-phatic cell.

The invention also features a pharmaceutical composition comprising the engineered, non-human immune cell describe above and a pharmaceutically acceptable carrier.

The cell and composition can be used in a method for treating a neoplasia or cancer in a subject in need thereof. In that case, the antigen-binding domain binds to an antigen on or associated with a neoplasia or cancer. This method includes administering to the subject a therapeutically effective amount of the engineered, non-human immune cell or pharmaceutical composition. The antigen-binding domain binds to an antigen on a cancer cell or an antigen labeled onto a tumor. Examples of the cancer include hematological cancer, breast cancer, ovarian cancer, gastric cancer, prostate cancer, squamous cell carcinoma, head and neck cancer, colon cancer, pancreatic cancer, uterine cancer, renal cell cancer, glioblastoma, medulloblastoma, sarcoma, and lung cancer.

The cell and composition can also be used in a method for treating an infectious disease in a subject in need thereof. In that case, the antigen-binding domain binds to an antigen on or associated with an infectious pathogen, such as pathogenic parasite, bacterium, or virus. This method includes administering to the subject a therapeutically effective amount of the engineered, non-human immune cell or pharmaceutical composition.

The cell and composition can also be used in a method for treating an autoimmune disease in a subject in need thereof. In that case, the antigen-binding domain binds to a self-antigen or an antigen on or associated with an autoreactive lymphocyte. This method includes administering to the subject a therapeutically effective amount of the engineered, non-human immune cell or pharmaceutical composition.

The details of one or more embodiments of the invention are set forth in the description below. Other features, objectives, and advantages of the invention will be apparent from the description and from the claims.

### BRIEF DESCRIPTION OF THE DRAWING

FIG. 1 is a diagram showing an approach to select genetic modifications that reprogram the immune functions of murine T cells for better therapeutic purpose in a human host.

### DETAILED DESCRIPTION OF THE INVENTION

This invention relates to engineered immune cells derived from non-human vertebrate animals as a source for the manufacture of adoptive cellular immunotherapy such as CAR-based cell therapy. Specifically, these animals and/or their cells are engineered to (1) express natural antigen receptors or chimeric antigen receptors that recognize state-of-art immune therapeutic antigen targets and elicit desired effector responses; (2) minimize host-vs-graft reaction; (3) minimize graft-vs-host reaction; (4) obtain similar traffic capability as human immune cells; (5) obtain synthetic biological control of cellular functions; and (6) eliminate potential hazards such as the production of human sensitive infectious reagents. Immune cells such as T cells, B cells, NK cells, Macrophages etc. collected from those animals can, with or without expansion, be provided to patients in need to treat their corresponding diseases such as cancer, autoimmune diseases, or infection through adoptive cell therapy approach.

Compared with convention CAR-based cell therapies, the cells and therapies described herein allow one to improve the quality consistency and decease production cost of chimeric antigen receptor based immune cell therapy (e.g., CAR-T in cancer therapy). Combining xenotransplantation and cell based immune therapy, the invention here provides a method to produce CAR based immune cell therapy with lower cost, easier scale-up, and better consistency. With the invention disclosed herein, there is no need to introduce CAR-T vector into T cells ex vivo (a step imposes the major cost in reagents and labor) because all engineered nonhuman animal derived immune cells carry chimeric antigen receptors in their genome. Also, manufacture can start from non-human animals with defined genetic background and more stringent quality control standard, which will ensure high consistency of production and high quality. Furthermore, it is easy to scale as nonhuman animals such as livestock, can be scaled up or down organically according to needs.

### I. Engineered Immune Cells

The present invention provides engineered immune cells and methods for treating cancer, autoimmune diseases, or infection, among other diseases. In one embodiment, the invention provides an engineered immune cell (e.g., T cell) engineered to express a CAR wherein the cell exhibits an antitumor property or an anti-pathogen property. The cells can also be engineered so that they (i) express various therapeutic/beneficial factors (e.g., one or more different CARs, pro-function factors, and graft sustaining factors) and (ii) lack the expression of various harmful factors (e.g., rejection factors, host targeting factors, and infection-risk factor) as described herein. Various sources of non-human immune cells can be used to make the engineered immune cells in this invention.

### Sources of Cells

Prior to expansion and/or genetic modification of the immune cells of the invention, a source of immune cells can be obtained from a non-human subject. For example, T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available in the art, may be used. These cells can be engineered so that they express the therapeutic/beneficial factors (e.g., CARs, pro-function factors, and graft sustaining factors) and lack the expression of various harmful factors (e.g., rejection factors, host targeting factors, and infection-risk factor) as described herein.

Alternatively, the engineered immune cells of this invention can be derived from a stem cell or obtained from a genetically engineered non-human animal that has been already engineered to express the various therapeutic/beneficial factors (e.g., CARs, pro-function factors, and graft sustaining factors) and lack the expression of various harmful factors (e.g., rejection factors, host targeting factors, and infection-risk factor) as described herein.

Various genomic editing technologies such as homologue recombination, TALEN, CRISPR/Cas, and Zinc Finger endonucleases enabled large-scale genome modification of immune cells, stem cells, or genetically engineered non-human animal. In some embodiments, the factors described herein can be introduced simultaneously through a specific genome editing method or a combination of multiple technologies. The genomic engineering elements can be delivered into an immune cell, a stem cell, or a fertilized germline cell through electroporation, liposome, polymers, viral vectors, or microinjection. In some other embodiments, the factors described herein are introduced to an engineered non human immune cell derived from genetically engineered host animal through stepwise editing. A subgroup of factors is engineered into a germline cell of a host animal use a genomic editing technology or a combination. A germline cell from an embryo or an animal with correctly edited genome of the first subgroup of factors will be used to carry on further genomic editing of the second group of factors. A cycle of 2, 3, 4, 5-10, 10-20, or 20-50, will be used to achieve final product of engineered immune cells, stem cells, germline cells, or a genetically engineered animal carrying all desired genomic editions. Techniques for genome modification of immune cells, stem cells, or genetically engineered non-human animal are known in the art. See e.g., WO2012079000, US20170152526 and US20170275362.

The germline cell can be a primary embryonic stem cell, an induced pluripotent stem cells (iPS), an embryonic stem cell line (ES cells), or a pluripotent cell that can give rise to multiple daughter cell types.

### T Cell Antigen Receptors

Various T cell antigen receptors (TCRs) can be expressed in the cells of this invention. In some embodiments, a TCR recognizes tumor antigen or tumor associate antigens such as NY-ESO-1, MART-1, or IDH. In some other embodiments, a TCR recognizes antigenic peptide derived from infectious reagents such as HIV, HPV, or Flu. In some other embodiments, a TCR recognizes antigenic peptide derived from self-antigens related too autoimmune diseases such as insulin, myelin basic protein. The TCR can be natural isolated gene from human subjects or an engineered TCR with increased affinity with same antigen specificity.

### CAR

Various CARs can be expressed in cells of this invention. To date, there are three generations of CARs. The “First generation” CARs are typically composed of an extracellular antigen-binding domain (e.g., a single-chain variable fragment fused to a transmembrane domain, fused to cytoplasmic/intracellular domain of a T cell receptor chain. “First generation” CARs typically have the intracellular domain from the CD3ζ-chain, which is the primary transmitter of signals from endogenous TCRs. “First generation” CARs can provide de novo antigen recognition and cause activation of both CD4⁺ and CD8⁺ T cells through their CD3ζ chain signaling domain in a single fusion molecule, independent of HLA-mediated antigen presentation. “Second generation” CARs add intracellular domains from various co-stimulatory molecules (e.g., CD28, 4-1BB, ICOS, OX40) to the cytoplasmic tail of the CAR to provide additional signals to the T cell. “Second generation” CARs comprise those that provide both co-stimulation (e.g., CD28 or 4-1BB) and activation (CD34 Preclinical studies have indicated that “Second Generation” CARs can improve the anti tumor activity of T cells. For example, robust efficacy of “Second Generation” CAR modified T cells was demonstrated in clinical trials targeting the CD19 molecule in patients with chronic lymphoblastic leukemia (CLL) and acute lymphoblastic leukemia (ALL). “Third generation” CARs comprise those that provide multiple co-stimulation (e.g., CD28 and 4-1BB) and activation (CD3ζ) See e.g., Cell. 2017; 168(4):724-740, Nat Rev Cancer. 2016; 16(9):566-81.

In accordance with the presently disclosed subject matter, the CARs comprise among others an extracellular antigen-binding domain, a transmembrane domain and an intracellular domain, where the extracellular antigen-binding domain binds to an antigen of interest. In a specific non-limiting embodiment, the extracellular antigen-binding domain is a scFv. In a specific non-limiting embodiment, the extracellular antigen-binding domain is a Fab, which is optionally crosslinked. In a specific non-limiting embodiment, the extracellular binding domain is an F(ab)₂. In a specific non-limiting embodiment, any of the foregoing molecules may be comprised in a fusion protein with a heterologous sequence to form the extracellular antigen-binding domain.

Antigen Binding Moiety

The CAR of the invention can comprise a target-specific binding element or an antigen-binding moiety. The choice of moiety depends upon the type and number of ligands that define the surface of a target cell. For example, the antigen-binding domain may be chosen to recognize a ligand that acts as a cell surface marker on target cells associated with a particular disease state. Thus examples of cell surface markers that may act as ligands for the antigen moiety domain in the CAR of the invention include those associated with viral, bacterial and parasitic infections, autoimmune disease and cancer cells,

In one embodiment, the CAR can be engineered to target a tumor antigen of interest by way of engineering a desired antigen binding moiety that specifically binds to an antigen on a tumor cell. In the context of the present invention, “tumor antigen,” “antigen on a tumor” or “antigen associated with a hyperproliferative disorder” refers to antigens that are common to specific hyperproliferative disorders such as neoplasia, tumor or cancer.

These antigens include proteins or other biological molecules that are produced by tumor cells that elicit an immune response, particularly T-cell mediated immune responses. The selection of the antigen-binding moiety of the invention will depend on the particular type of cancer to be treated. Tumor antigens are well known in the art and include, for example, a glioma-associated antigen, carcinoembryonic antigen (CEA), β-human chorionic gonadotropin, alphafetoprotein (AFP), lectin-reactive AFP, thyroglobulm, RAGE-1, MN-CA IX, human telomerase reverse transcriptase, RU1, RU2 (AS), intestinal carboxyi esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin,

In one embodiment, the antigen comprises one or more antigenic cancer epitopes associated with a malignant tumor. Malignant tumors express a number of proteins that can serve as target antigens for an immune attack. These molecules include but are not limited to tissue-specific antigens such as MART-1, tyrosinase and GP100 in melanoma and prostatic acid phosphatase (PAP) and prostate-specific antigen (PSA) in prostate cancer. Other target molecules belong to the group of transformation-related molecules such as the oncogene HER-2/Neu ErbB-2. Yet another group of target antigens is onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B-cell differentiation antigens such as CD19, CD20, BCMA, CD138, and CD37 are other candidates for target antigens in B-cell lymphoma. Some of these antigens (CEA, HER-2, CD19, CD20, idiotype) have been used as targets for passive immunotherapy with monoclonal antibodies.

The type of tumor antigen useful in the invention may also be a tumor-specific antigen (TSA) or a tumor-associated antigen (TAA). A TSA is unique to tumor cells and does not occur on other cells in the body. A TAA associated antigen is not unique to a tumor cell and instead is expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development when the immune system is immature and unable to respond or they may be antigens that are normally present at extremely low levels on normal cells but which are expressed at much higher levels on tumor cells.

Non-limiting examples of TSA or TAA antigens include: Differentiation antigens such as MART-1/MelanA (MART-1), gp100 (Pmel 17), tyrosinase, TRP-1, TRP-2 and tumor-specific multi-lineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, 1GH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H 1, PSA, TAG-72, CA 19-9, CA 72-4, CAM 17.1, NuMa, K-ras, beta-Catenin, CDK4, Mum-1, p15, p16, 43-9F, 5T4 (791Tgp72} alpha-fetoprotem, beta-HCG, BCA225, BTAA, CA-125, CA 15-3\CA 27.29\BCAA, CA 195, CA 242, CA-50, CAM43, CD68\PI, CO-029, FGF-5, G250, Ga733\EpCAM, HTgp-175, M344, MA-50, MG7-Ag, MOV18, NB/70K, NY-CO-1, RCAS1, SDCCAG16, TA-90Mac-2 binding protein\cyclophilin C-associated protein, TAAL6, TAG72, TLP, and TPS.

In a preferred embodiment, the antigen binding moiety portion of the CAR targets an antigen that includes but is not limited to CD19, CD20, CD22, ROR 1, Mesothelin, CD33/lL3Ra, c-Met, PSMA, Glycolipid F77, EGFRvIII, GD-2, NY-ESO-1, MAGE-A3, and the like.

Depending on the desired antigen to be targeted, the CAR of the invention can be engineered to include the appropriate antigen bind moiety that is specific to the desired antigen target. For example, if CD19 is the desired antigen that is to be targeted, an antibody for CD 19 can be used as the antigen bind moiety for incorporation into the CAR of the invention. In another example, if HIV infection is to be treated, a broad neutralizing antibody against the HIV envelope glycoprotein (GP120) can be used as the antigen bind moiety for incorporation into the CAR of the invention (Mol Ther. 2017 Mar. 1; 25(3):570-579). In the other example, if an auto-reactive antibody generating B cells is the target to treat autoimmune disease, a self-antigen that bind to the auto-reactive B cells can be used as the antigen bind moiety for incorporation into the CAR of the invention (e.g. Science. 2016 Jul. 8; 353(6295):179-84).

Transmembrane Domain

With respect to the transmembrane domain, the CAR can be designed to comprise a transmembrane domain that is fused to the extracellular domain of the CAR. In one embodiment, the transmembrane domain that naturally is associated with one of the domains in the CAR is used. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein. Transmembrane regions of particular use in this invention may be derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154. Alternatively the transmembrane domain may be synthetic, in which case it will comprise predominantly hydrophobic residues such as leucine and valine. Preferably, a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

Cytoplasmic Domain

The cytoplasmic domain (or the intracellular signaling domain) of the CAR of the invention is responsible for activation of at least one of the normal effector functions of the immune cell in which the CAR has been placed in. The term “effector function” refers to a specialized function of a cell. Effector function of a T cell, for example, may be cytolytic activity or helper activity including the secretion of cytokines. Thus, the term “intracellular signaling domain” refers to the portion of a protein that transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Preferred examples of intracellular signaling domains for use in the CAR of the invention include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

It is known that signals generated through the TCR alone may be insufficient for full activation of the T cell and that a secondary or co-stimulatory signal may be required. Thus, T cell activation can be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

Primary cytoplasmic signaling sequences regulate primary activation of the TCR complex either in a stimulatory way, or in an inhibitory way. Primary cytoplasmic signaling sequences that act in a stimulatory manner may contain signaling motifs that are known as immunoreceptor tyrosine-based activation motifs or ITAMs.

Examples of ITAM containing primary cytoplasmic signaling sequences that are of particular use in the invention include those derived from TCR zeta, FcR gamma, FcR beta, CD3 gamma, CD3 delta, CD3 epsilon, CD5, CD22, CD79a, CD79b, and CD66d. It is particularly preferred that cytoplasmic signaling molecule in the CAR of the invention comprises a cytoplasmic signaling sequence derived from CD3 zeta.

In a preferred embodiment, the cytoplasmic domain of the CAR can be designed to comprise the CD3-zeta signaling domain by itself or combined with any other desired cytoplasmic domain(s) useful in the context of the CAR of the invention. For example, the cytoplasmic domain of the CAR can comprise a CD3 zeta chain portion and a costimulatory signaling region. The costimulatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like. Thus, while the invention in exemplified primarily with 4-1BB as the co-stimulatory signaling element, other costimulatory elements are within the scope of the invention.

The cytoplasmic signaling sequences within the cytoplasmic signaling portion of the CAR of the invention may be linked to each other in a random or specified order. Optionally, a short oligo- or polypeptide linker, preferably between 2 and 10 amino acids in length may form the linkage. A glycine-serine doublet provides a particularly suitable linker.

In one embodiment, the cytoplasmic domain is designed to comprise the signaling domain of CD3-zeta and the signaling domain of CD28. In another embodiment, the cytoplasmic domain is designed to comprise the signaling domain of CD3-zeta and the signaling domain of 4-IBB. In yet another embodiment, the cytoplasmic domain is designed to comprise the signaling domain of CD3-zeta and the signaling domain of CD28 and 4-IBB.

### Pro Function Factors

As disclosed herein, the engineered non-human immune cells of this invention express other therapeutic/beneficial factors in addition to CAR. Examples of other therapeutic/beneficial factors include human derived or humanized pro-function factors. A pro-function factor refers to factor that can enable biological functions towards a cell targeted by the CARs. Examples of the pro-function factor include perforin, granzyme B, pro-inflammatory cytokines, integrins, and chemokines.

Perforin is a pore forming cytolytic protein found in the granules of cytotoxic T lymphocytes (CTLs) and NK cells. Upon degranulation, perforin binds to the target cell's plasma membrane, and oligomerises in a Ca²⁺ dependent manner to form pores on the target cell. The pore formed allows for the passive diffusion of a family of pro-apoptotic proteases, known as the granzymes, into the target cell. The lytic membrane-inserting part of perforin is the MACPF domain. This region shares homology with cholesterol-dependent cytolysins from Gram-positive bacteria. Perforin has structural and functional similarities to complement component 9 (C9). Like C9, this protein creates transmembrane tubules and is capable of lysing non-specifically a variety of target cells. This protein is one of the main cytolytic proteins of cytolytic granules, and it is known to be a key effector molecule for T-cell- and natural killer-cell-mediated cytolysis. Perforin is thought to act by creating holes in the plasma membrane that triggers an influx of calcium and initiates membrane repair mechanisms. These repair mechanisms bring perforin and granzymes into early endosomes.

Granzyme B is a serine protease most commonly found in the granules of CTLs, NK cells, and cytotoxic T cells. It is secreted by these cells along with the pore forming protein perforin to mediate apoptosis in target cells. Granzyme B has shown to be involved in inducing inflammation by stimulating cytokine release and is also involved in extracellular matrix remodelling. Once inside the target cell granzyme B can cleave and activate initiator caspases 8 and 10, and executioner caspases 3 and 7, which trigger apoptosis. Granzyme B can also cleave BID leading to BAX/BAK oligomerisation and cytochrome c release from the mitochondria. Granzyme B can cleave ICAD leading to DNA fragmentation and the laddering pattern associated with apoptosis. Granzyme B can also generate a cytotoxic level of mitochondrial reactive oxygen species (ROS) to mediate cell death.

A pro-inflammatory cytokine (or inflammatory cytokine) is a type of signaling molecule that is normally excreted from immune cells like helper T cells (Th) and macrophages, and certain other cell types that promote inflammation. They include interleukin-1 (IL-1), IL-12, and IL-18, tumor necrosis factor (TNF), interferon gamma (IFN-gamma), and granulocyte-macrophage colony stimulating factor and play an important role in mediating the innate immune response. Inflammatory cytokines are predominately produced by and involved in the upregulation of inflammatory reactions.

Integrins are transmembrane receptors that facilitate cell-extracellular matrix (ECM) adhesion. Upon ligand binding, integrins activate signal transduction pathways that mediate cellular signals such as regulation of the cell cycle, organization of the intracellular cytoskeleton, and movement of new receptors to the cell membrane. The presence of integrins allows rapid and flexible responses to events at the cell surface that facilitates the formation of the stable conjugation between T cells and target cells essential for optimal T immune cell activation, proliferation, cytokine production, and killing.

Chemokines are a family of small cytokines, or signaling proteins, which can induce directed chemotaxis in nearby responsive cells. Chemokines are classified into four main subfamilies: CXC, CC, CX3C and XC. All of these proteins exert their biological effects by interacting with G protein-linked transmembrane receptors called chemokine receptors, that are selectively found on the surfaces of their target cells (Mélik-Parsadaniantz, Stephane; Rostène, William (2008). “Chemokines and neuromodulation”. Journal of Neuroimmunology. 198 (1-2): 62-8). As chemokines can control cells of the immune system during processes of immune surveillance, such as directing lymphocytes to the lymph nodes so they can screen for invasion of pathogens by interacting with antigen-presenting cells residing in these tissues, the engineered immune cells of this invention can also direct a host's lymphocytes to a cell or site via CAR-mediated targeting. In that case, such inflammatory chemokines function mainly as chemoattractants for leukocytes, recruiting monocytes, neutrophils and other effector cells from the blood to targeted sites. Certain inflammatory chemokines activate cells to initiate an immune response or promote wound healing. They can serve to guide cells of both innate immune system and adaptive immune system. Both homeostatic chemokines and inflammatory chemokines can be used in this invention. Homeostatic chemokines are responsible for basal leukocyte migration and include CCL14, CCL19, CCL20, CCL21, CCL25, CCL27, CXCL12 and CXCL13. Inflammatory chemokines actively participate in the inflammatory response attracting immune cells to the site of inflammation and examples include CXCL-8, CCL2, CCL3, CCL4, CCL5, CCL11, and CXCL10.

Other examples of the pro-function factor include neutralizing antibodies against immune suppressive factors including anti-PD-1, anti-PD-L1, and anti-CTLA-4 etc., immune costimulatory molecule agonist including anti-4-1BB, anti-OX40, anti-CD40L etc. dominant negative receptors, metabolic enzymes.

### Graft Sustaining Factors

Other examples of the therapeutic/beneficial factors include graft sustaining factors. A graft sustaining factor is an agent or a factor that can reduce or suppress host rejection thereby prolong graft survival. Xenotransplantation research has identified key factors that can prolong graft survival for weeks to months. Examples of graft sustaining factors include human complement-regulatory protein, CTLA4, CTLA4-Ig, PD-L1, a mutant form of human MHC class II, some human MHC class I molecules either wild type or mutant form such as HLA-E, HLA-G, HLA Cw3, CD47, HO-1, ATPase or thrombomdulin.

The above-described factors have therapeutic effect or protect the engineered non human cells' survival once infused in a human subject. Accordingly, the engineered non human cells can be genetically modified to express one or more of such factors or contain a nucleic acid encoding such a factor.

The engineered non-human cells of this invention can also be genetically modified to eliminate or modify expression of one or more of factors that are not conducive to the cells or the host patient. These factors include rejection factors, host targeting factors, and infection-risk factor as described below.

### Rejection Factors

A rejection factor refers to a molecular, a moiety on the molecule, or a factor that is itself recognized by a host immune response or generating factors recognized by the host immune response on the cell surface. Examples of rejection factor include galactose-α1,3-galactose, and N-glycolylneuraminic acid.

Galactose-alpha-1,3-galactose, commonly known as alpha gal, is a carbohydrate found in most mammalian cell membranes. It is not found in primates, including humans, whose immune systems recognize it as a foreign body and produce xenoreactive immunoglobulin M antibodies, leading to organ rejection after transplantation. N-Glycolylneuraminic acid (Neu5Gc) is a sialic acid molecule found in most non-human mammals. Humans are known to have natural antibodies against Neu5Gc. Galactose-alpha-1,3-galactose and N-Glycolylneuraminic acid can be eliminated from the engineered non human cells of this invention through the disruption of one or more key enzymes synthesizing these modifications such as N-acetylgalactosaminyltransferase, α1,3-galactosyltransferase, CMP-N-acetylneuraminic acid hydroxylase. Accordingly, the engineered non-human cells of this invention can lack one or more of these enzymes. See e.g., Cooper D K C, et al. Xenotransplantation. 2016: 23: 83-105, Cowan P J, et al. (June 2000). Transplantation. 69 (12): 2504-15, and Lutz et al. Xenotransplantation 2013; 20: 27-35.

### Host Targeting Factors

A host targeting factor refers to a factor that can recognize normal or nonmalignant human cells/tissues. Examples of this factor include a T cell receptor that recognize human tissues through cross species activity. This factor should be eliminated or modified in the engineered immune cell through the disruption of endogenous T cell receptor genes such as murine-TCR-alpha by various genomic editing or RNA technologies (such as those based on gene knockout, TALEN, CRISPR/Cas, Zinc Finger endonucleases, and RNAi) or through re-expressing of TCR with known antigenic specificity that will not recognize human tissues such as OT-1 See e.g., C57BL/6-Tg(TcraTcrb)1100Mjb/J, The Jackson Laboratory.

### Infection-Risk Factors

An infection-risk factor refers to a human sensitive infectious reagent that is harbored in the donor animal genome. Examples include various endogenous retrovirus. In some embodiments, when an immune cell from porcine is to be engineered for adoptive cellular immunotherapy for human, porcine endogenous retroviruses (PERVs) in normal porcine genome can be activated to produce viral particles infecting a bystander human cell. CRISPR/Cas system and other state-of-art genomic modification procedure in the manner described above can also be used to eliminate viral elements such as the reverse transcriptase gene (pol). See e.g., Science. 2015; 350(6264):1101-4. In some other embodiments, the viral receptors for viruses with cross species infectivity such as the receptor for influenza are eliminated from donor non-human immune cells.

### II. Formulations

The engineered, non-human immune cell of this invention and compositions comprising the cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the compositions comprising engineered, non-human immune cells of this invention in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE,” 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, suitable antibiotics, parabens, chlorobutanol, phenol, sorbic acid, and the like. Prolonged absorption of the injectable pharmaceutical form can be brought about by the use of agents delaying absorption, for example, alum inurn monostearate and gelatin. According to the present invention, however, any vehicle, diluent, or additive used would have to be compatible with the engineered, non-human immune cell of this invention.

The compositions can be isotonic, i.e., they can have the same osmotic pressure as blood and lacrimal fluid. The desired isotonicity of the compositions of the presently disclosed subject matter may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Viscosity of the compositions, if desired, can be maintained at the selected level using a pharmaceutically acceptable thickening agent. Methylcellulose can be used because it is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. The choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Those skilled in the art will recognize that the components of the compositions should be selected to be chemically inert and will not affect the viability or efficacy of the engineered, non-human immune cell of this invention. This will present no problem to those skilled in chemical and pharmaceutical principles, or problems can be readily avoided by reference to standard texts or by simple experiments (not involving undue experimentation), from this disclosure and the documents cited herein.

One consideration concerning the therapeutic use of the engineered, non-human immune cells of this invention is the quantity of cells necessary to achieve an optimal effect. The quantity of cells to be administered will vary for the subject being treated. In certain embodiments, from about 10⁴ to about 10¹⁰, from about 10⁵ to about 10⁹, or from about 10⁶ to about 10⁸ engineered, non-human immune cells of this invention are administered to a subject. More effective the cells may be administered in even smaller numbers. In certain embodiments, at least about 1×10⁸, about 2×10⁸, about 3×10⁸, about 4×10⁸, and about 5×10⁸ cells of the invention are administered to a human subject. The precise determination of what would be considered an effective dose may be based on factors individual to each subject, including their size, age, sex, weight, and condition of the particular subject. Dosages can be readily ascertained by those skilled in the art from this disclosure and the knowledge in the art.

The skilled artisan can readily determine the amount of cells and optional additives, vehicles, and/or carrier in compositions and to be administered in methods of the presently disclosed subject matter. Typically, any additives (in addition to the active cell(s) and/or agent(s)) can be present in an amount of from about 0.001% to about 50% by weight) solution in phosphate buffered saline, and the active ingredient can be present in the order of micrograms to milligrams, such as from about 0.0001 wt % to about 5 wt %, from about 0.0001 wt % to about 1 wt %, from about 0.0001 wt % to about 0.05 wt %, from about 0.001 wt % to about 20 wt %, from about 0.01 wt % to about 10 wt %, or from about 0.05 wt % to about 5 wt %. For any composition to be administered to an animal or human, and for any particular method of administration, toxicity should be determined, such as by determining the lethal dose (LD) and LD50 in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

### III. Method of Treatments

The present invention provides cells and methods for treating various diseases, including cancer. The cancer may be a hematological malignancy, a solid tumor, a primary or a metatastizing tumor. Preferably, the cancer is a hematological malignancy, and more preferably, the cancer is Chronic Lymphocytic Leukemia (CLL). Other diseases treatable using the compositions and methods of the invention include viral, bacterial and parasitic infections as well as autoimmune diseases.

In some cases, the engineered cells may be used to treat a neoplasia or a cancer, including solid tumors and hematologic malignancies. Non-limiting examples of cancers include: acute lymphoblastic leukemia, acute myeloid leukemia, adrenocortical carcinoma, AIDS-related cancers, AIDS-related lymphoma, anal cancer, appendix cancer, astrocytomas, neuroblastoma, basal cell carcinoma, bile duct cancer, bladder cancer, bone cancers, brain tumors, such as cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas, Burkitt lymphoma, carcinoma of unknown primary origin, central nervous system lymphoma, cerebellar astrocytoma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma, germ cell tumors, gallbladder cancer, gastric cancer, gastrointestinal carcinoid tumor, gastrointestinal stromal tumor, gliomas, hairy cell leukemia, head and neck cancer, heart cancer, hepatocellular (liver) cancer, Hodgkin lymphoma, Hypopharyngeal cancer, intraocular melanoma, islet cell carcinoma, Kaposi sarcoma, kidney cancer, laryngeal cancer, lip and oral cavity cancer, liposarcoma, liver cancer, lung cancers, such as non-small cell and small cell lung cancer, lymphomas, leukemias, macroglobulinemia, malignant fibrous histiocytoma of bone/osteosarcoma, medulloblastoma, melanomas, mesothelioma, metastatic squamous neck cancer with occult primary, mouth cancer, multiple endocrine neoplasia syndrome, myelodysplastic syndromes, myeloid leukemia, nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer, ovarian germ cell tumor, pancreatic cancer, pancreatic cancer islet cell, paranasal sinus and nasal cavity cancer, parathyroid cancer, penile cancer, pharyngeal cancer, pheochromocytoma, pineal astrocytoma, pineal germinoma, pituitary adenoma, pleuropulmonary blastoma, plasma cell neoplasia, primary central nervous system lymphoma, prostate cancer, rectal cancer, renal cell carcinoma, renal pelvis and ureter transitional cell cancer, retinoblastoma, rhabdomyosarcoma, salivary gland cancer, sarcomas, skin cancers, skin carcinoma merkel cell, small intestine cancer, soft tissue sarcoma, squamous cell carcinoma, stomach cancer, T-cell lymphoma, throat cancer, thymoma, thymic carcinoma, thyroid cancer, trophoblastic tumor (gestational), cancers of unknown primary site, urethral cancer, uterine sarcoma, vaginal cancer, vulvar cancer, Waldenstrm macroglobulinemia, and Wilms tumor.

In some cases, an engineered cell of the disclosure may be used to treat an infectious disease. An infectious disease may be caused, for example, by a pathogenic bacterium or by a virus. Various pathogenic proteins, nucleic acids, lipids, or fragments thereof can be expressed by a diseased cell. An antigen presenting cell can internalize such pathogenic molecules, for instance with phagocytosis or by receptor-mediated endocytosis, and display a fragment of the antigen bound to an appropriate MHC molecule. For instance, various 9 mer fragments of a pathogenic protein may be displayed by an APC. Engineered cells of the disclosure may be designed to recognize various antigens and antigen fragments of a pathogenic bacterium or a virus. Non-limiting examples of pathogenic bacteria can be found in the: a) Bordetella genus, such as Bordetella pertussis species; b) Borrelia genus, such Borrelia burgdorferi species; c) Brucelia genus, such as Brucella abortus, Brucella canis, Brucella meliterisis, and/or Brucella suis species; d) Campylobacter genus, such as Campylobacter jejuni species; e) Chlamydia and Chlamydophila genus, such as Chlamydia pneumonia, Chlamydia trachomatis, and/or Chlamydophila psittaci species; f) Clostridium genus, such as Clostridium botulinum, Clostridium difficile, Clostridium perfringens, Clostridium tetani species; g) Corynebacterium genus, such as Corynebacterium diphtheria species; h) Enterococcus genus, such as Enterococcus faecalis, and/or Enterococcus faecium species; i) Escherichia genus, such as Escherichia coli species; j) Francisella genus, such as Francisella tularensis species; k) Haemophilus genus, such as Haemophilus influenza species; l) Helicobacter genus, such as Helicobacter pylori species; m) Legionella genus, such as Legionella pneumophila species; n) Leptospira genus, such as Leptospira interrogans species; o) Listeria genus, such as Listeria monocytogenes species; p) Mycobacterium genus, such as Mycobacterium leprae, Mycobacterium tuberculosis, and/or Mycobacterium ulcerans species; q) Mycoplasma genus, such as Mycoplasma pneumonia species; r) Neisseria genus, such as Neisseria gonorrhoeae and/or Neisseria meningitidia species; s) Pseudomonas genus, such as Pseudomonas aeruginosa species; t) Rickettsia genus, such as Rickettsia rickettsii species; u) Salmonella genus, such as Salmonella typhi and/or Salmonella typhimurium species; v) Shigella genus, such as Shigella sonnei species; w) Staphylococcus genus, such as Staphylococcus aureus, Staphylococcus epidermidis, and/or Staphylococcus saprophyticus species; x) Streptpcoccus genus, such as Streptococcus agalactiae, Streptococcus pneumonia, and/or Streptococcus pyogenes species; y) Treponema genus, such as Treponema pallidum species; z) Vibrio genus, such as Vibrio cholera; and/or aa) Yersinia genus, such as Yersinia pestis species.

In some cases, an engineered cell of the disclosure may be used to treat an infectious disease, an infectious disease may be caused a virus. Non-limiting examples of viruses can be found in the following families of viruses and are illustrated with exemplary species: a) Adenoviridae family, such as Adenovirus species; b) Herpesviridae family, such as Herpes simplex type 1, Herpes simplex type 2, Varicella-zoster virus, Epstein-barr virus, Human cytomegalovirus, Human herpesvirus type 8 species; c) Papillomaviridae family, such as Human papillomavirus species; d) Polyomaviridae family, such as BK virus, JC virus species; e) Poxviridae family, such as Smallpox species; f) Hepadnaviridae family, such as Hepatitis B virus species; g) Parvoviridae family, such as Human bocavirus, Parvovirus B19 species; h) Astroviridae family, such as Human astrovirus species; i) Caliciviridae family, such as Norwalk virus species; j) Flaviviridae family, such as Hepatitis C virus (HCV), yellow fever virus, dengue virus, West Nile virus species; k) Togaviridae family, such as Rubella virus species; l) Hepeviridae family, such as Hepatitis E virus species; m) Retroviridae family, such as Human immunodeficiency virus (HIV) species; n) Orthomyxoviridaw family, such as Influenza virus species; o) Arenaviridae family, such as Guanarito virus, Junin virus, Lassa virus, Machupo virus, and/or Sabia virus species; p) Bunyaviridae family, such as Crimean-Congo hemorrhagic fever virus species; q) Filoviridae family, such as Ebola virus and/or Marburg virus species; Paramyxoviridae family, such as Measles virus, Mumps virus, Parainfluenza virus, Respiratory syncytial virus, Human metapneumovirus, Hendra virus and/or Nipah virus species; r) Rhabdoviridae genus, such as Rabies virus species; s) Reoviridae family, such as Rotavirus, Orbivirus, Coltivirus and/or Banna virus species. In some examples, a virus is unassigned to a viral family, such as Hepatitis D.

In some cases, an engineered T-cell of the disclosure may be used to treat an immune disease, such as an autoimmune disease. Inflammatory diseases, including autoimmune diseases are also a class of diseases associated with B-cell disorders. Examples of immune diseases or conditions, including autoimmune conditions, that can be treated with an engineered cell disclosed herein include: rheumatoid arthritis, rheumatic fever, multiple sclerosis, experimental autoimmune encephalomyelitis, psoriasis, uveitis, diabetes mellitus, systemic lupus erythematosus (SLE), lupus nephritis, eczema, scleroderma, polymyositis/scleroderma, polymyositis/dermatomyositis, uncerative protitis, severe combined immunodeficiency (SCID), DiGeorge syndrome, ataxia-telangiectasia, seasonal allergies, perennial allergies, food allergies, anaphylaxis, mastocytosis, allergic rhinitis, atopic dermatitis, Parkinson's, Alzheimer's, hypersplenism, leukocyte adhesion deficiency, X-linked lymphoproliferative disease, X-linked agammaglobulinemia, selective immunoglobulin A deficiency, hyper IgM syndrome, HIV, autoimmune lymphoproliferative syndrome, Wiskott-Aldrich syndrome, chronic granulomatous disease, common variable immunodeficiency (CVID), hyperimmunoglobulin E syndrome, Hashimoto's thyroiditis, acute idiopathic thrombocytopenic purpura, chronic idiopathic thrombocytopenia purpura, dermatomyositis, Sydenham's chorea, myasthenia gravis, polyglandular syndromes, bullous pemphigoid, Henoch-Schonlein purpura, poststreptococcalnephritis, erythema nodosum, erythema multiforme, gA nephropathy, Takayasu's arteritis, Addison's disease, sarcoidosis, ulcerative colitis, polyarteritis nodosa, ankylosing spondylitis, Goodpasture's syndrome, thromboangitisubiterans, Sjogren's syndrome, primary biliary cirrhosis, Hashimoto's thyroiditis, thyrotoxicosis, chronic active hepatitis, polychondritis, pamphigus vulgaris, Wegener's granulomatosis, membranous nephropathy, amyotrophic lateral sclerosis, tabes dorsalis, giant cell arteritis, polymyalgia, peraiciousanemia, rapidly progressive glomerulonephritis, psoriasis, fibrosing alveolitis, and cancer.

One or multiple engineered cells disclosed herein can be administered to a subject in any order or simultaneously. If simultaneously, the multiple engineered cells can be provided in a single, unified form, such as an intravenous injection, or in multiple forms, for example, as multiple intravenous infusions, s.c, injections. The engineered cells can be packed together or separately, in a single package or in a plurality of packages. One or all of the engineered cells can be given in multiple doses. If not simultaneous, the timing between the multiple doses may vary to as much as about a week, a month, two months, three months, four months, five months, six months, or about a year. In some cases, an engineered cell can expand within a subject's body, in vivo, after administration to a subject. Engineered cells can be frozen to provide cells for multiple treatments with the same cell preparation. Engineered cells disclosed herein and pharmaceutical compositions comprising the same, can be packaged as a kit. A kit may include instructions (e.g., written instructions) on the use of the engineered cells and compositions.

Engineered cells disclosed herein may be formulated in unit dosage forms suitable for single administration of precise dosages. In some cases, the unit dosage forms can comprise additional other lymphocytes. In unit dosage form, the formulation can be divided into unit doses containing appropriate quantities of cells. The unit dosage can be in the form of a package containing discrete quantities of the formulation. Non-limiting examples are packaged capsules, vials, ampoules, bags, tubes, or syringes for injectors . . . etc. Aqueous suspension compositions can be packaged in single-dose non-re-closable containers. Multiple-dose re-closable containers can be used, for example, in combination with or without a protective/preservative agent. Formulations for parenteral injection can be presented in unit dosage form, for example, in ampoules, or in multi-dose containers with a protective/preservative agent.

In some embodiments, the engineer immune cells disclosed herein may be formulated in freezing media and placed in cryogenic storage units such as liquid nitrogen freezers (−195° C.) or ultra-low temperature freezers (−65° C., −80° C. or −120° C.) for long-term storage of at least about 1 month, 2 months, 3 months, 4 months, 5 months, 6 months, 1 year, 2 years, 3 years, or at least 5 years. The freeze media can contain dimethyl sulfoxide (DMSO), and/or sodium chloride (NaCl), and/or dextrose, and/or dextran sulfate and/or hydroyethyl starch (HES) with physiological pH buffering agents to maintain pH between about 6.0 to about 6.5, about 6.5 to about 7.0, about 7.0 to about 7.5, about 7.5 to about 8.0 or about 6.5 to about 7.5. The cryopreserved cells can be thawed and further processed by stimulation with antibodies, proteins, peptides, and/or cytokines as described herein. The cryopreserved cells can be thawed and further genetically modified with viral vectors (including retroviral and lentiviral vectors) or non-viral means (including RNA, DNA, and proteins) as described herein. The modified cells can be further cryopreserved to generate cell banks in quantities of at least about 1, 5, 10, 100, 150, 200, 500 vials at about at suitable density (e.g., at least 10¹, 10², 10³, 10⁴, 10⁵, 10⁶, 10⁷, 10⁸, 10⁹, or at least about 10¹⁰ cells per mL in freeze media. The cryopreserved cell banks may retain their functionality and can be thawed and further stimulated and expanded. In some aspects, thawed cells can be stimulated and expanded in suitable closed vessels such as cell culture bags and/or bioreactors to generate quantities of cells as allogeneic cell product. Cryopreserved cells can maintain their biological functions for at least about a long period (e.g., 1 month to 10 years) under cryogenic storage condition. In some aspects, no preservatives are used in the formulation. The cryopreserved cells can be thawed and infused into multiple patients as off-the-shelf cell product.

### IV. Definition

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the term “antibody” (Ab) is used in the broadest sense and specifically may include any immunoglobulin, whether natural or partly or wholly synthetically produced, including but not limited to monoclonal antibodies, polyclonal antibodies, multispecific antibodies (for example, bispecific antibodies and polyreactive antibodies), and antibody fragments. Thus, the term “antibody” as used in any context within this specification is meant to include, but not be limited to, any specific binding member, immunoglobulin class and/or isotype (e.g., IgG1, IgG2a, IgG2b, IgG3, IgG4, IgM, IgA1, IgA2, IgD, and IgE) and biologically relevant fragment or specific binding member thereof, including but not limited to Fab, F(ab′)2, scFv (single chain or related entity) and (scFv)₂.

As used herein, the term “antibody fragments” may include those antibody fragments obtained using techniques readily known and available to those of ordinary skill in the art, as reviewed herein. Therefore, in addition to the definition for “antibody” presented supra, the term “antibody” may further encompass any polypeptide or protein comprising a portion of an intact antibody, such as the antigen binding or variable region of the intact antibody. These can be derived from natural sources, or they may be partly or wholly synthetically produced. Examples of antibody fragments include, but are not limited to, Fab, Fab′, F(ab′)2, and Fv fragments; diabodies, and linear antibodies.

As used herein, the term “scFv” may refer to a single-chain variable fragment. scFv is a fusion protein of the variable regions of the heavy (VH) and light chains (VL) of immunoglobulins, connected with a linker peptide. The linker peptide can be from about 5 to 40 amino acids or from about 10 to 30 amino acids or about 5, 10, 15, 20, 25, 30, 35, or 40 amino acids in length.

As used herein, the term “Fab fragment” may refer to a recognition moiety that is a fragment of antigen binding fragment (Fab), equivalently the two arms of an antibody molecule, which is comprised of an integrated light chain and the VH and the CH1 domains of a heavy chain. As used herein, the term “Fc” refers to the “fragment crystallisable” region, which is comprised of the CH2 and CH3 constant domains and is an interacting region of immunoglobulin that interacts with effector molecules or cells, e.g. Fc receptors.

As used herein, the term “Fe receptor” or “FcR” is used to describe a receptor that binds to an Fc region (e.g. the Fc region of an antibody or antibody fragment). The term includes the neonatal receptor, FcRn, which is responsible for the transfer of maternal IgGs to the fetus. Other Fc receptors include any one of FcγRI, FcγRIIA, FcγRIIB1, FcγRIIB2, FcγRIIIA, FcγRIIIB, FccRI, FccRII, FcαRI, and Fcα/μR.

The term “antigen” or “Ag” as used herein is defined as a molecule that is targeted through intermolecular interaction on the cell surface by engineered immune receptors such as T cell receptors (TCR), Chimeric antigen receptors (CAR), or protein fusion based bio-functional receptor on a engineered non-human immune cell. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, the term “carriers” may include pharmaceutically acceptable carriers, excipients, or stabilizers that are nontoxic to the cell or mammal being exposed thereto at the dosages and concentrations employed. Often the pharmaceutically acceptable carrier is an aqueous pH buffered solution. Examples of physiologically acceptable carriers include, but not limited to, buffers such as phosphate, citrate, and other organic acids; antioxidants including, but not limited to, ascorbic acid; low molecular weight (less than about 10 residues) polypeptide; proteins, such as, but not limited to, serum albumin, gelatin, or immunoglobulins; hydrophilic polymers such as, but not limited to, polyvinylpyrrolidone; amino acids such as, but not limited to, glycine, glutamine, asparagine, arginine or lysine; monosaccharides, disaccharides, and other carbohydrates including, but not limited to, glucose, mannose, or dextrins; chelating agents such as, but not limited to, EDTA; sugar alcohols such as, but not limited to, mannitol or sorbitol; salt-forming counterions such as, but not limited to, sodium; and/or nonionic surfactants such as, but not limited to, TWEEN; polyethylene glycol (PEG), and PLURONICS. Any combination of such components, including probable inclusion of a bacteriostat, may be useful to fill the formulations of the present disclosure.

The terms “specific binding,” “selective binding,” “selectively binds,” and “specifically binds,” may refer to antibody binding to an epitope on a predetermined antigen but not to other antigens. Typically, the antibody binds with an equilibrium dissociation constant (K_{D}) of approximately less than 10⁻⁶ M, such as approximately less than 10⁻⁷ M, 10⁻⁸ M, 10⁻⁹ M or 10⁻¹⁰ M or even lower when determined by, e.g., equilibrium dialysis or surface plasmon resonance (SPR) technology in a BIACORE® 2000 surface plasmon resonance instrument using the predetermined antigen as the analyte and the antibody as the ligand, or Scatchard analysis of binding of the antibody to antigen positive cells, and (ii) binds to the predetermined antigen with an affinity that is at least two-fold greater than its affinity for binding to a non-specific antigen (e.g., BSA, casein) other than the predetermined antigen or a closely-related antigen.

As used herein, the terms “subject” or “patient” may refer to a biological system to which a treatment can be administered. A biological system can include, for example, an individual cell, a set of cells (e.g., a cell culture), an organ, a tissue, or a multi-cellular organism. A subject of the present invention may include birds, reptiles, mammals and the like. Preferably, the mammal comprises rodents and primates, including human. Subjects can be humans, non-human primates such as chimpanzees, and other apes and monkey species; farm animals such as cattle, horses, sheep, goats, swine or pig; domestic animals such as rabbits, dogs, and cats; laboratory animals including rodents, such as rats, mice and guinea pigs, and the like. A subject can be of any age. Subjects can be, for example, elderly adults, adults, adolescents, pre-adolescents, children, toddlers, infants.

As used herein, the term “treating” or “treatment” of a disease may refer to executing a protocol, which may include administering one or more drugs to a patient (human or otherwise), in an effort to alleviate signs or symptoms of the disease. Alleviation can occur prior to signs or symptoms of the disease appearing as well as after their appearance. Thus, “treating” or “treatment” includes “preventing” or “prevention” of disease. In addition, “treating” or “treatment” does not require complete alleviation of signs or symptoms, does not require a cure, and specifically includes protocols that may have only a marginal effect on the patient.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc, of the subject to be treated. In certain embodiments, an “effective amount” is an amount sufficient to arrest, ameliorate, or inhibit the continued proliferation, growth, or metastasis (e.g., invasion, or migration) of a neoplasia or cancer.

As used herein, the term “neoplasia” refers to a disease characterized by the pathological proliferation of a cell or tissue and its subsequent migration to or invasion of other tissues or organs. Neoplasia growth is typically uncontrolled and progressive, and occurs under conditions that would not elicit, or would cause cessation of, multiplication of normal cells. Neoplasias can affect a variety of cell types, tissues, or organs, including but not limited to an organ selected from the group consisting of bladder, colon, bone, brain, breast, cartilage, glia, esophagus, fallopian tube, gallbladder, heart, intestines, kidney, liver, lung, lymph node, nervous tissue, ovaries, pleura, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasias include cancers, such as sarcomas, carcinomas, or plasmacytomas (malignant tumor of the plasma cells).

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

The term “autoimmune disease” as used herein is defined as a disorder that results from an autoimmune response. An autoimmune disease is the result of an inappropriate and excessive response to a self-antigen. Examples of autoimmune diseases include but are not limited to, Addision's disease, alopecia greata, ankylosing spondylitis, autoimmune hepatitis, autoimmune parotitis, Crohn's disease, diabetes (Type 1), dystrophic epidermolysis bullosa, epididymitis, glomerulonephritis, Graves' disease, Guillain-Barr syndrome, Hashimoto's disease, hemolytic anemia, systemic lupus erythematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, myxedema, pernicious anemia, ulcerative colitis, among others.

A “nucleic acid” refers to a DNA molecule (e.g., a cDNA or genomic DNA), an RNA molecule (e.g., an mRNA), or a DNA or RNA analog. A DNA or RNA analog can be synthesized from nucleotide analogs. The nucleic acid molecule can be single-stranded or double-stranded, but preferably is double-stranded DNA. An “isolated nucleic acid” is a nucleic acid the structure of which is not identical to that of any naturally occurring nucleic acid or to that of any fragment of a naturally occurring genomic nucleic acid. The term therefore covers, for example, (a) a DNA which has the sequence of part of a naturally occurring genomic DNA molecule but is not flanked by both of the coding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. The nucleic acid described above can be used to express CARs or factors described herein. For this purpose, one can operatively linked the nucleic acid to suitable regulatory sequences to generate an expression vector.

A vector refers to a nucleic acid molecule capable of transporting another nucleic acid to which it has been linked. The vector can be capable of autonomous replication or integrate into a host DNA. Examples of the vector include a plasmid, cosmid, or viral vector. The vector of this invention includes a nucleic acid in a form suitable for expression of the nucleic acid in a host cell. Preferably the vector includes one or more regulatory sequences operatively linked to the nucleic acid sequence to be expressed. Examples of a “regulatory sequence” include promoters, enhancers, and other expression control elements (e.g., polyadenylation signals). Regulatory sequences also include those that direct constitutive expression of a nucleotide sequence, as well as tissue-specific regulatory and/or inducible sequences. The design of the expression vector depends on such factors as the choice of the host cell to be introduced in, the level of expression of protein desired, and the like. The expression vector can be introduced into host cells to produce the CARs or factors described herein.

### EXAMPLES

### Example 1

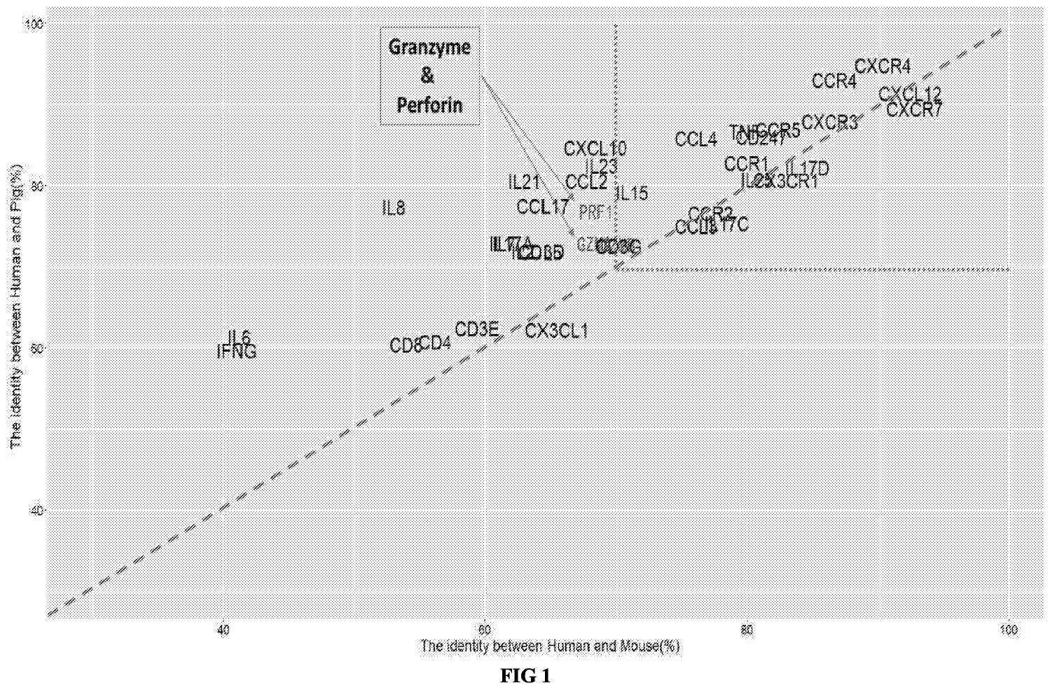

One approach to select the genetic modifications that reprogram the immune functions of the murine T cells for better therapeutic purpose in the human host including the following:
1. Collect gene and protein sequences of CD247, CD3G, CD3E, CD3D, IL-2, IFN-G, IL-6, IL7, IL-8, IL-15, IL-17A, IL-17C, IL-17D, IL21, IL23, IL25, CD4, CD8, PRF1, GZMB, GZMA, TNF, CCL2, CCL3, CCL4, CCR1, CCR2, CCR5, CCL5, CCR5, CCL17, CCR4, CXCL10, CXCR3, CXCL12, CXCR4, CXCR7, CX3 CL 1, CX3 CR 1 from Mus musculus, Sus scrofa, and, Homo sapiens;
2. Conduct sequence alignments between species against Homo sapiens to obtain cross species sequence identity for individual genes;
3. As shown in FIG. 1, the immune molecules with the donor sequence identity lower than 70% from Homo sapiens are selected to be engineered to integrate corresponding human forms into the non-human donor lymphocytes to improve functional efficacy in the human host.

### Example 2

Stepwise genomic engineering to obtain a murine derived non-human immune cell targeting CD19 molecules on the human B cell lymphoma for adoptive cellular immunotherapy is carried out as following:
1) A mouse embryonic stem cell #1 (mESC #1) is derived from OT-1 TCR transgenic mouse (C57BL/6-Tg(TcraTcrb)1100Mjb/J) under standard protocol (Nat Protoc. 9(3): 559-574);
2) Three small guide RNA (sgRNA) oligonucleotides targeting mouse endogenous retrovirus (MERV) pol gene on the genome with different sequence specificity are pre-loaded on to Cas9 protein and the complexes are then transfected into the mESC from step 1. A mESC colony is selected in which a) the gene knockout is verified both genetically by droplet digital PCR (ddPCR) and functionally by co-culture with human HEK-293 without detectable MERV in the human cells, and b) no detectable off-target gene editing events through whole genome sequencing;
3) A homozygous mouse is generated from the confirmed genomic edited mESC in step 2 with confirmed OT-1 TCR transgenic expression and the lack of all endogenous retroviral pol genes;
4) A mouse embryonic stem cell #2 (mESC #2) is derived from the mouse in step 3 using the same protocol as in step 1;
5) Three small guide RNA (sgRNA) oligonucleotides with different sequence specificity per gene targeting mice Rag1 (Gene ID: 19373), mice N-acetylgalactosaminyltransferase (Gene ID: 108148), mice CMP-N-acetylneuraminic acid hydroxylase Gene ID: 12763), and mice α1,3 galactosyltransferase (Gene ID: 396733) the genome are pre-loaded on to Cas9 protein and the complexes are then transfected into the mESC from step 4. A mESC colony is selected in which a) the gene knockout of all 4 targets is confirmed by droplet digital PCR (ddPCR), and b) no detectable off-target gene editing events through whole genome sequencing;
6) A homozygous mouse is generated from the confirmed genomic edited mESC colony in step 5 with confirmed OT-1 TCR transgenic expression and the lack of all endogenous retroviral pol genes, mice Rag1, mice CMP-N-acetylneuraminic acid hydroxylase, α1,3-galactosyltransferase, and N-acetylgalactosaminyltransferase;
7) A mouse embryonic stem cell #3 (mESC #3) is derived from the mouse in step 6 using the same protocol as in step 1;
8) A BAC containing human HLA-E (Gene ID: 3133), human perforin (Gene ID: 5551), human Granzyme B (Gene ID: 3002), human CD47 (Gene ID: 961), human CD55 (Gene ID: 1604) coding sequences under MMLV promoter is prepared to be integrated into mice genome;
9) The construct from step #8 is injected into the mESC from step 7;
10) A mouse homozygous breading pair expressing OT-1 TCR transgene, human HLA-E, human perforin, human Granzyme B, human CD47, and human CD55 with confirmed knockout of all endogenous retroviral pol genes, mice Rag1, mice CMP-N-acetylneuraminic acid hydroxylase, α1,3-galactosyltransferase, and N-acetylgalactosaminyltransferase is obtained from the mESC from step 9 and standard mice genetic breading strategy;
11) A mouse embryonic stem cell #4 (mESC #4) is derived from the mouse in step 10 using the same protocol as in step 1;
12) A transgene construct containing the chimeric antigen receptor (CAR) composing of the anti-human CD19scFv as the antigen binding domain, transmembrane domain of mice CD28 as the transmembrane domain, and the intracellular domain of mice CD28 followed by mice TCRζ chain as the activation domain using techniques known in the art (e.g., WO2012079000, US20170152526 and US20170275362). The construct is then injected into the mESC from step 11 and genomic integration is confirmed through standard genetic test;
13) A mouse homozygous breading pair expressing anti-human CD19 CAR (described in step 12), OT-1 TCR transgene, human HLA-E, human perforin, human Granzyme B, human CD47, and human CD55 with confirmed knockout of all endogenous retroviral pol genes, mice Rag1, mice CMP-N-acetylneuraminic acid hydroxylase, α1,3-galactosyltransferase, and N-acetylgalactosaminyltransferase is obtained from the mESC from step 12 and standard mice genetic breading strategy;
14) Mature CD8+ T cells from an animal bred from step 13 are obtained from lymph nodes, spleens, or blood and further expanded by anti-murine CD3/CD28 microspheres.
15) Expanded anti-human CD19 CAR-T cells from step 14 are counted, formulated, functionally tested, sterility tested, aliquoted, and stored till they are needed for patient infusion;

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the scope of the invention, and all such variations are intended to be included within the scope of the following claims. All references cited herein are incorporated by reference in their entireties.

1. An engineered, non-human immune cell, comprising an antigen-binding receptor or a nucleic acid encoding the antigen-binding receptor, wherein the engineered, non-human immune cell has one or more of the following features:
(a) expressing a pro-function factor or comprising a nucleic acid encoding the pro-function factor;
(b) lacking expression of an rejection factor;
(c) expressing a graft sustaining factor or comprising a nucleic acid encoding the graft sustaining factor;
(d) eliminating or modifying the expression of a host targeting factor, and
(e) lacking the activity of an infection-risk factor.

2. The engineered, non-human immune cell of claim 1, wherein the antigen-binding receptor is a T cell antigen receptor (TCR) or a chimeric antigen receptors (CAR), said TCR or CAR comprising an antigen binding domain, a transmembrane domain, and an activation domain.

3. The engineered, non-human immune cell of claim 1, wherein the pro-function factor is selected from the group consisting of human or humanized perforin, granzyme B, and pro-inflammatory cytokines.

4. The engineered, non-human immune cell of claim 1, wherein the rejection factor is galactose-α1,3-galactose or N-glycolylneuraminic acid (NeuGc).

5. The engineered, non-human immune cell of claim 1, wherein the graft sustaining factor is human complement-regulatory protein, CTLA4, CTLA4-Ig, PD-L1, a mutant form of human MHC class II, a wild type or mutant form of human MHC class I, CD47, HO-1, ATPase or thrombomdulin.

6. The engineered, non-human immune cell of claim 5, wherein the human complement-regulatory protein is CD55, CD46, or CD59.

7. The engineered, non-human immune cell of claim 1, wherein an endogenous antigen receptor or activating receptor that recognizes a health human tissue is eliminated, modified or replaced.

8. The engineered, non-human immune cell of claim 1, wherein one or more key molecules controlling the activity of human sensitive infectious reagents are eliminated for safety.

9. The engineered, non-human immune cell of claim 1, being derived from a non-human animal.

10. The engineered, non-human immune cell of claim 1, being a CD4 or CD8 T cell, a Gamma-delta T cell, a NKT cell, a B cell, a NK cell, or a macrophage.

11. The engineered, non-human immune cell of claim 1 is engineered through germline modification, somatic modification, or ex vivo.

12. The engineered, non-human immune cell of claim 2, wherein the antigen binding domain is an antibody, an antigen-binding fragment thereof, or a ligand of the antigen.

13. The engineered, non-human immune cell of claim 12, wherein the antigen-binding fragment is a Fab or a scFv.

14. The engineered, non-human immune cell of claim 13, wherein the antigen binding domain binds to an antigen on a tumor or an antigen labeled onto a tumor.

15. The engineered, non-human immune cell of claim 14, wherein the antigen is associated with a hematologic malignancy.

16. The engineered, non-human immune cell of claim 15, wherein the antigen is associated with a solid tumor.

17. A pharmaceutical composition comprising the engineered, non-human immune cell of claim 1 and a pharmaceutically acceptable carrier.

18. A method for treating a neoplasia or a cancer in a subject in need thereof, comprising administering to the subject a therapeutically effective amount of the engineered, non-human immune cell of claim 1, wherein the antigen binding domain binds to an antigen on a cancer cell or an antigen labeled onto a tumor.

19. The method of claim 18, wherein the cancer is selected from the group consisting of hematological cancer, breast cancer, ovarian cancer, gastric cancer, prostate cancer, squamous cell carcinoma, head and neck cancer, colon cancer, pancreatic cancer, uterine cancer, renal cell cancer, glioblastoma, medulloblastoma, sarcoma, and lung cancer.

20. A method for treating an infectious disease in a subject in need thereof, comprising administering to the subject a therapeutically effective amount of the engineered, non-human immune cell of claim 1, wherein the antigen binding domain binds to an antigen from an infectious pathogen.

21. A method for treating an autoimmune disease in a subject in need thereof, comprising administering to the subject a therapeutically effective amount of the engineered, non-human immune cell of claim 1, wherein the antigen binding domain binds to a self-antigen or an antigen on an autoreactive lymphocyte.
